(19) United States
(12) Patent Application Publication
(10) Pub. No.: US 2021/0330587 A1
(43) Pub. Date: Oct. 28, 2021

(54) ORAL SANITIZER AND IMMUNE SUPPORT FOR VIRAL AND BACTERIAL PREVENTION

(71) Applicant: COVIGUARD CORP, MIAMI, FL (US)

(72) Inventors: LISA MARIE KAO, MIAMI, FL (US); FRANKLIN GARCIA-GODOY, CORDOVA, TN (US)

(21) Appl. No.: 17/243,520

(22) Filed: Apr. 28, 2021

Related U.S. Application Data
(60) Provisional application No. 63/016,444, filed on Apr. 28, 2020.
(60) Provisional application No. 63/086,317, filed on Oct. 1, 2020.

(51) Int. Cl. A61K 9/00; A61K 47/02; A61K 47/10; A61K 47/12; A61K 47/40; A61K 45/06

(52) U.S. Cl. CPC A61K 9/0056; A61K 9/0078; A61K 47/02; A61K 45/06; A61K 47/12; A61K 47/40; A61K 47/10

(57) ABSTRACT

An oral sanitizer composition with immune support for viral, bacterial and COVID-19 prevention. The oral sanitizer composition formulated into a mouthwash, a mouth rinse, a mouth strip a throat lozenge, a mouth spray, a throat spray or a nasal spray. The oral sanitizer composition being formulated with alcohol and without alcohol.

### RELATED APPLICATIONS

This applicant claims the benefit of U Provisional Application Ser. No. 63/016,444 filed Apr. 28, 2020 and Se. No. 63/086,317 filed Oct. 1, 2020, each of which is incorporated herein by reference in its entirety.

### FIELD OF THE INVENTION

The claimed invention relates to an antibacterial and antiviral prevention with immune support, particularly in an oral hygiene and nasal composition. More particularly, the claimed invention in a form of a mouth rinse, mouth spray, throat and sublingual spray, nasal spray, mouth-strips and Lozenges.

### BACKGROUND OF THE INVENTION

Coronavirus Disease 2019 (COVID-19) is a respiratory disease caused by the SARS-CoV-2 virus. The Covid-19 spread from China to many other countries around the world, including the United States. Depending on the severity of COVID-19's international impacts, outbreak conditions—including those rising to the level of pandemic—can affect all aspects of daily life, including travel, trade, tourism, food supplies, and financial markets. COVIDd-19, infection with SARS- CoV-2, the virus that causes COVID-19, can cause mild and severe illness, in some cases, can be fatal. Symptoms typically include fever, cough, and shortness of breath. Certain people infected with the virus have reported experiencing other non-respiratory symptoms. Still certain other people, referred to as asymptomatic cases, have experienced no symptoms at all. According to the Centers for Disease Control and Prevention (CDC), symptoms of COVID-19 may appear in few as 2 days or as long as 14 days after exposure.

Although the first human cases of COVID-19 likely resulted from exposure to infected animals, infected people can spread SARS-CoV-2 to other people. The virus is thought to spread mainly from person-to-person, including: between people who are in close contact with one another (within about 6 feet) through respiratory droplets produced when an infected person coughs or sneezes. These droplets can land in the mouths and noses of people who are nearby or possibly be inhaled into the lungs.

The current ways of prevention are washing hands, using hand sanitizers, wearing masks, exercising social distance, i.e., 6 feet, covering coughs and sneezes. However, the current methodology does not address reducing the viral load to prevent the spread of COVID-19. The claimed invention proceeds on the desirability of addressing these deficiencies.

Therefore, it is an object of the claimed invention, to prevent airborne transmission of viral and bacterial pathogens, such as COVID-19, entering people's mouth and nasal passages through respiratory tract while supporting their immune system. Presently, there is no solution for this ongoing problem. The claimed invention minimizes and/or prevents transmission and spread of COVID-19 and other viral and airborne bacterial pathogens.

### Oral Cavity Disease Transmission of COVID-19

The COVID-19 (or 2019-CoV) epidemic attributed to an emerging coronavirus SARS-CoV-2 in December 2019 has generated severe threats to international security, global health, and the global economy. Given current lack of effective treatment for COVID-19, there is a need to explore other alternative methods to contain the propagation of these infections, focusing particular on its mode of transmission. The person-to-person modes of transmission of SARS-CoV-2 are direct transmissions, such as sneezing, coughing, transmission through inhalation of small droplets, and transmission by contact, such as contact with nasal, oral, and ocular mucous membranes. SARS-CoV-2 can also be transmitted directly or indirectly by the saliva. Other possible route of person-to-person transmission can include the fetal-oral routes. Moreover, high viral loads have been found in the oropharynx of infected patients, as well as in the asymptomatic subjects. This could suggest that the potential of SARS-CoV-2 transmission is wider than originally thought. The oral cavity is therefore directly associated with the evolutionary process of SARS-CoV-2 in its inhalation of ambient particles in the air and in expectorations.

Considered to be a major portal of entry for infectious agents, the oral cavity is colonized by a large number and variety of micro-organisms, including bacteria, fungi, and viruses termed microbiota. These microbial communities and their inevitable multiple synergistic and antagonistic interactions are reflected in an individual's oral and general health. The oral cavity and nasopharyngeal regions can be considered as the anatomical transition between external and internal environments. The standard oral cavity temperature is on average 37° C. with no notable variations, which gives the microorganisms a secure environment to survive.

Saliva is also pH stable at 6.5-7, which is a favorable pH to oral microbiota (bacteria species and virus, such as coronavirus). Variations in saliva composition are often associated with microbiota dysbiosis and oral diseases. Moreover, salivary composition influences oropharyngeal colonization characteristics and bacterial profile. Oral microbiota comprises commensal bacterial populations that sustain mutual benefits with the host and keep potentially pathogenic bacteria in balance through a number of negative feedback mechanisms. In the oral biofilm, these bacteria combine to make a barrier resistant to antibiotics, disinfectants, mechanical removal, and other stresses. In addition, bacteria within biofilms have 1000 times more resistance to antibacterial treatments compared to planktonic microorganisms.

In the absence of proper oral hygiene, the percentage of potentially health damaging bacteria in biofilm increases, contributing to the development of chronic infection. Moreover, in biofilms, bacteria can escape the immune system producing so-called superantigens In addition to host-microbe interactions, the interfaces of periodontal pathogens with other non-host pathogens, such as herpesviruses, like Epstein-Barr virus and cytomegalovirus, can contribute to the pathogenesis of the periodontal disease, or can affect the outcome of viral infection and dissemination. These risks are often significantly underestimated. Accordingly, oral care measures that are effective in reducing infection should be given higher priority because the entry point for influenza infection is the mucosal tissue on the roof of the mouth, as determined by National Institutes of Health (NIH).

### OBJECT AND SUMMARY OF THE INVENTION

There are two types of mouthwash: cosmetic and therapeutic. Cosmetic mouthwash temporarily controls bad breath and leaves behind a pleasant taste, but has no chemical or biological application beyond their temporary benefit. For example, if a product does not kill bacteria associated with bad breath, then its benefit is considered to be solely cosmetic. Therapeutic mouthwash, by contrast, has active ingredients intended to help control or reduce conditions like bad breath, gingivitis, plaque, and tooth decay. There is no mouthwash, mouth rinse or mouth spray currently on the market that is formulated to help prevent viral, bacterial and/or COVID-19 spread in an oral hygiene application.

The object of the claimed invention is to prevent airborne transmission of viral and bacterial pathogens such as COVID-19, while supporting the immune system. The claimed invention is provided in the form of an oral sanitizer, mouth-rinse, mouth-spray, lozenges, throat and sublingual spray, mouth strips and nasal spray, collectively referred to herein as the “antibacterial and antiviral preventive rinse and/or spray.”

Considered to be a major portal of entry for infectious agents, the oral cavity is directly associated with the evolutionary process of SARS-CoV-2 in its inhalation of ambient particles in the air and in expectorations. New generations of mouth rinses and mouth and nasal sprays are needed in the market and should have ingredients that contribute to lowering the SARS-CoV-2 viral load, thereby facilitating the fight against oral transmission. Chlorhexidine, a typical component of a mouth rinse, is not efficient in killing SARS-CoV-2. Hence, in accordance with an exemplary embodiment of the claimed invention, the claimed mouth rinses and/or local nasal applications comprising β-cyclodextrins combined with flavonoids agents, such as citrus bioflavanoid 50% HPLC, provide valuable adjunctive treatment in reducing the viral load of saliva and nasopharyngeal microbiota, including potential SARS-CoV-2 carriage. Preventive effects of βCD-Citrus Bioflavanoid 50% HPLC therapeutic oral biofilm rinses reduce the viral and bacterial load of the infection disease progression. In accordance with an exemplary embodiment of the claimed invention, the claimed mouth rinses comprise vitamin C, zinc and Citrus Bioflavanoids 50% HPLC, which act as adjuncts to help support the immune system.

The claimed mouth rinses and COVID-19-specific treatment, in the absence of vaccines or medicines that will unfortunately arrive too late for many patients, will be crucial in providing coronavirus specific interventions. The preventive mouth rinses and mouth sprays in accordance with the claimed invention can contribute to the reduction of the SARS-CoV-2 viral load, thereby facilitating the fight against oral transmission.

In accordance with an exemplary embodiment of the claimed invention, an oral sanitizer composition with immune support for viral, bacterial and COVID-19 prevention is provided. The composition comprises: 0% to 0.50% by weight of polysorbate 20; 0% to 10% by weight of Xylitol; 0% to 5% by weight methylated β-cyclodextrin; 0% to 10.50% by weight of vitamin C with citrus bioflavonoids; 0% to 10% by weight of sucralose; 0% to 0.10% by weight potassium sorbate; 0% to 0.006% by weight of sodium benzoate; 0% to 5% by weight of xanthan gum; 0% to 5% by weight of menthol; 0% to 5% by weight of zinc chloride; 0% 5% by weight of zinc masker; 0% to 10% by weight of FDC Green Color; and a remaining percentage by weight of water to total 100%.

In accordance with an exemplary embodiment of the claim invention, the oral sanitizer composition comprises: 0% to 0.50% by weight of potassium sorbate; 0% to 10% by weight poloxamer 407; 0% to 5% by weight of a buffering agent capable of buffering the oral sanitizer composition to a pH of 3.0 to 8.0; 0% to 10.5% by weight of a glycerin; 0% to 10% by weight of xylitol or sucralose; 0% to 0.10% by weight of zinc chloride; 0% to 0.006% by weight of FDC Green 3; 0% to 5% by weight of methylated beta cyclodextrins; 0% to 10% citrus bioflavonoids; 0% to 0.50% by weight of zinc chloride; 0% to 10% by weight of vitamin C; 0% to 25% by weight of ethanol 190 proof 98.05; 0% to 0.295% by weight of thymol; 0% to 0.1972% by weight of menthol; 0% to 0.438% by weight of methyl salicylate; 0% to 0.5663% by weight of eucalyptus oil; and a remaining percentage by weight of water to total 100%.

In accordance with an exemplary embodiment of the claimed invention, the oral sanitizer composition aforesaid is formulated into a mouthwash, a mouth rinse, a mouth strip or a throat lozenge.

In accordance with an exemplary embodiment of the claimed invention, the oral sanitizer composition aforesaid is formulated into a mouth spray, a throat spray or a nasal spray.

In accordance with an exemplary embodiment of the claimed invention, the oral sanitizer composition aforesaid is deliverable via a meter dose spray pump.

### DETAILED DESCRIPTION OF THE EMBODIMENT OF THE INVENTION

In accordance with the claimed invention, mouth rinses, mouth sprays, nasal sprays, mouth strips and lozenges comprise cyclodextrins, citrus bioflavanoids 50% HPLC and additional immune supporting ingredients for CoVID-19-Specific preventive treatment. The claimed compositions can be in any form common in the art, e.g., gel, aerosol, lozenge, mouth strip and the like and can also be formulated into systems for use in dual-compartment type dispensers.

The use of the claimed mouth rinses and nasal applications comprising cyclodextrins and citrus bioflavonoids 50% HPLC provide a valuable adjunct treatment. They are locally administered delivery systems that lower the SARS-CoV-2 viral load and reduce the nasopharyngeal microbiota, which tends to coat the surface of the aerosol particles and droplets during coughing or sneezing. In addition, the claimed composition comprising vitamin C, zinc and citrus bioflavanoids 50% HPLC also act as adjuncts to help support immune system. Preventive effects of βCD-Citrus Biofvanoids 50% HPLC therapeutic oral biofilm rinses are reducing the viral load of the infection and disease progression to upper respiratory tract.

Cyclodextrins (CDs) are natural derivatives of glucose, with a rigid cyclic structure, composed of α(1-4)-linked glucopyranoside units. The most usual CDs, called, α, β, and γ, contain 6-, 7-, and 8-glucopyranoside units, respectively. CDs are cyclic oligosaccharides used for improving bioavailability of medicinal products and water-solubility. Also, CDs can be employed to prevent or reduce ocular and gastrointestinal irritation, decrease or eliminate disagreeable tastes or smells, and prevent interactions between drugs or drug additives in a formulation. They have been used in a multitude of commercial sectors, such as deodorants, drug delivery, food, and cosmetics. On the European market, examples of use of CDs in drugs are y-CD in a minoxidil solution, β-CD in cetirizine tablets and cisapride suppositories. Examples of use of β-CD derivatives are HP-β-CD in itraconazole antifungal, in intravenous and oral solutions, SBE-β-CD in intravenous voriconazole antimycotic, and RM-β-CD in a nasal spray for hormone replacement therapy with 17β-estradiol. Cyclodextrins have many advantages. They are more biocompatible than most oxides contained in oral products (i.e., silver and gold) and simpler to use. They do not generate a resistance reaction and they are not toxic. Cyclodextrins have no harmful effects and are considered “generally regarded as safe” for humans. The fields of pharmaceutical application of CDs are notable due to their low immunogenicity, low toxicity, cost-effectiveness, and accessibility. These applications include but not limited to: increasing drug stability and solubility, improving drug absorption, masking undesirable tastes and odors, controlling drug release, eliminating local and systemic toxicity, and improving drug permeability via biological barriers.

The structure of cyclodextrins can be modified and used for containment of infections or as virucidal agents. For example, it has been established that methylated beta-cyclodextrin can reduce influenza A virus and coronavirus infectivity by sequestering cholesterol from viral particles or depleting it from the host cell membranes. Hydroxypropyl-βcyclodextrin was used as a vaccine adjuvant providing protection against the H1N1 influenza virus. One point to emphasize is that for natural CDs, the intramolecular hydrogen bond with the CD molecule decreases their hydrogen bond formation with the surrounding water molecules. This could lead to a potential negative antiviral action against the coronavirus.

Modifying the cyclodextrins with mercaptoundecane sulfonic acids provides a key nontoxic virucidal action and mimics heparan sulfates. Very promising studies have indicated that the modified sugar molecules attract viruses before irreversibly inactivating them. By disrupting the outer shell of a virus, cyclodextrin modified with mercaptoundecane sulfonic acids can destroy infectious particles by simple contact, rather than simply blocking viral growth. This mechanism seems to be the same regardless of the virus concerned. These modified cyclodextrins are biocompatible, broad-spectrum, and virucidal at in vitro micromolar concentrations against many viruses, including respiratory syncytial virus (RSV), herpes simplex virus (HSV), Zika virus, and dengue virus. They are effective ex vivo against both laboratory and clinical strains of RSV and HSV-2 in respiratory.

In accordance with an exemplary embodiment of the claimed invention, citrus bioflavanoids act as an antimicrobial and adds immune support whose components are based on soluble bio flavonoids derived from citrus fruits. Bioflavonoids are hydroylated phenolic structures synthesized by plants and have previously been shown to have activity against bacteria, fungi and viruses.

Pre-rinse mouth rinses with COVID-19-specific pre-rinse dental treatment has been recognized as aid for viral prevention in the dental community when treating patients and recognized by the ADA as an adjunct to help as a preventive measure when treating patients in dental chair settings. Use of pre-procedural rinse of peroxide is recommended. The concentration of any rinse used should be at least 0.5%. Over-the-counter peroxide is typically 2%, so it would need to be diluted. CDA and ADA recommend rinsing at the beginning of the appointment for 60 seconds and again after the appointment. The CDC recommends cleaning surfaces in the home with the peroxide.

In accordance with an exemplary embodiment of the claimed invention, an antibacterial and anti-viral mouthwash is provided that is physiologically acceptable to the users. Antibacterial mouthwashes of the claimed invention are effective in inhibiting microorganisms found in the mouth, thus preventing caries and ameliorating infectious conditions.

In accordance with an exemplary embodiment of the claimed invention, the immune supporting ingredients comprises citrus bioflavnoids 50% HPLC, zinc, goldenseal and vitamin C. The citrus bioflavonoids 50% HPLC in the claimed immune supporting ingredients provide immune support for the respiratory tract. Zinc in the claimed immune supporting ingredients, which is found in cells throughout the body, helps the immune system fight off invading bacteria and viruses, and treats the common cold. Goldenseal in the claimed immune supporting ingredients is a potent antiviral medication and found to inhibit the growth of the H1N1 influenza virus in human cells. Vitamin C in the claimed immune supporting ingredients shortens the duration of the common cold and ameliorate symptom severity in the general population due to the anti-histamine effects of high-dose vitamin C. Preferably, the claimed immune supporting ingredients comprising 3000 mg of vitamin C is suggested for patients with COVID-19.

In accordance with an exemplary embodiment of the claimed invention, the immune supporting ingredients comprise liquid vitamin supplements which can be absorbed under the tongue and entering the bloodstream directly without having to go through the gastrointestinal tract. Additionally, sublingual vitamins have many other benefits. Since the vitamins and nutrients in the claimed immune supporting ingredients are absorbed through the tongue, they are quickly released into the body and their nutrients are not broken down by the stomach acid. As a result, the claimed immune supporting ingredients with the sublingual vitamins can be taken in smaller dosages, and are easier and more convenient to take than oral vitamins.

In accordance with an exemplary embodiment of the claimed invention, the composition of the claimed antibacterial and antiviral preventive rinse and/or spray with immune support comprises:
(a) from about 0.0 to 0.50 by weight of sodium benzoate;
(b) from about 0.01% to 0.50% by weight Xanthum Gum
(c) from about 2.0% to 5.0% by weight of a buffering agent capable of buffering the composition to a pH of 3.0 to about 8.0;
(d) from 0% to about 10.5% by weight of a glycerin;
(e) from 0% to about 5.0% by weight of a sweetening agent sorbitol or xylitol;
(f) from 0% to about 0.006% by weight of a flavoring agent zinc masker;
(g) 0.006 FDC Green 3;
(h) sufficient water to total 100%;
(i) from 0% to about 5.0% Beta Cyclodextrins;
(j) from 0% to about 5.0% Citrus Bioflavonoids 50% HPLC
(k) from 0% 50 mg Zinc Chloride;
(l) from 0% to 3000 mg Vitamin C;
(m) from 0% to Ethanol 190 proof 98.05; and
(n) from 0% to Blend of Thymol 0.295%, Menthol 0.1972, Methyl Salicylate 0.4348 Eucalyptus Oil 0.5663.

In accordance with an exemplary embodiment of the claimed invention, a method of inhibiting oral microorganisms comprises contacting the tissue of oral cavity or teeth with the claimed antibacterial and antiviral preventive rinse for a sufficient time to reduce the microorganisms and harmful pathogens of virus.

In accordance with an exemplary embodiment of the claimed invention, a method of controlling bacteria and virus in the mouth comprises contacting the tissue of oral cavity with the claimed antibacterial and antiviral preventive rinse for a sufficient time to reduce the bacteria and viral content while additionally providing immune supporting vitamins.

In accordance with an exemplary embodiment of the claimed invention, the composition of the claimed antibacterial and antiviral preventive rinse and/or spray with immune support comprises sweetening agents to sweeten the taste of the composition. In accordance with an aspect of the claimed invention, the sweeteners are sorbitol and xylitol. When a sweetening agent other than sodium saccharin is used in the composition of the claimed antibacterial and antiviral preventive rinse and/or spray with immune support, any amount required to produce an equivalent level of sweetening to the 0% to about 0.2% sodium saccharin will suffice. Additionally, any mixture of sweetening agents having an equivalent sweetening effect and compatible to the formulation is contemplated within the term of sweetening agents.

In accordance with an exemplary embodiment of the claimed invention, the composition of the claimed antibacterial and antiviral preventive rinse and/or spray with immune support comprises flavoring agents. Preferably, the flavoring agent is present in the composition at a concentration of from 0% to about 2.0% by weight of the total. More preferably, the concentration should be of from about 0.05% to about 2.0% with the most desired level being about 0.25%. In accordance with an exemplary embodiment of the claimed invention, the flavoring agents are selected from menthol, peppermint oil, spearmint oil, and other known flavor modifiers. Particularly, the preferred selection of the flavoring agents is peppermint, spearmint oil (both natural and synthetic analog), and a mixture of the two.

The composition of the claimed antibacterial and antiviral preventive rinse and/or spray with immune support additionally comprises water to serve as a fluid base of the composition and to function as a flushing medium to wash away food debris from the mouth.

The composition of the claimed antibacterial and antiviral preventive rinse and/or spray with immune support is a clear, stable, physiologically acceptable, and produces microorganisms inhibition in the mouth. Prolonged contact time will increase the effects, and it is preferred that the contact time be about 30-60 seconds.

In accordance with exemplary embodiment of the claimed invention, the composition of the claimed antibacterial and antiviral preventive rinse and/or spray with immune support is an oral hygiene composition.

In accordance with exemplary embodiment of the claimed invention, the oral hygiene composition is an oral sanitizer with immune support for viral, bacterial and COVID-19 prevention in mouthwash and spray form:
a) anti-bacterial and anti-viral formula, kills 99.9% by reducing viral load;
b) physiologically acceptable, weakens and kills virus in less than 30 seconds;
c) liquid and powder immune supporting ingredients;
d) weakens the virus membrane and kills the virus in liquid and aerosol spray form;
e) safe and organoleptically tolerable in the oral cavity and has no significant side effects either orally or systemically; and
f) controls malodor and the viral load of pathogens in the mouth when it contacts the tissue of the oral cavity for a sufficient time to reduce the malodor, which also reduces the risk of COVID and other potentially harmful virus and viral pathogens comprising an effective antibacterial combination consisting essentially of:
   (1) from about 0.05% to about 0.2% by weight, based on the total weight of the composition, of sodium benzoate;
   (2) from 0% to Ethanol 190 proof 98.05;
   (3) from 0% to Blend of Thymol 0.295%, Menthol 0.1972, Methyl Salicylate 0.4348, Eucalyptus Oil 0.5663.

Research suggests a link between poor oral hygiene and severity of Covid-19 which stresses the importance of having a healthy mouth with less pathogenic bacteria.

### Throat Lozenges

In accordance with an exemplary embodiment of the claimed invention, the claimed antibacterial and antiviral preventive rinse and/or spray with immune support is in the form of throat lozenges. A throat lozenge (also known as a cough drop, troche, cachou, pastille or cough sweet) is a small, typically medicated tablet intended to be dissolved slowly in the mouth to temporarily stop coughs, lubricate, and soothe irritated tissues of the throat (usually due to a sore throat), possibly from the common cold or influenza. Cough tablets have taken the name lozenge, based on their original shape, a diamond. The claimed throat lozenge with anti-viral and immune support helps in preventing transmission in oral cavity.

In accordance with an exemplary embodiment of the claimed invention, the claimed lozenges comprise beta cyclodextrins and citrus bioflavanoid 50% HPLC with zinc, thymol and eucalyptol, thereby providing anti-viral and anti-bacterial prevention. Whereas, typical lozenges contain benzocaine, an anesthetic, or eucalyptus oil. The claimed lozenges help in reducing or suppressing flu and cold. Additionally, claimed lozenges reduce viral load in mouth, thereby reducing the spread to respiratory tract and cases infection.

In accordance with an exemplary embodiment of the claimed invention, the lozenges comprise particles including but not limited to beta cyclodextrin and a binder. The average surface area mass ratio of the particles is equal to or greater than 1 square meter per gram (m²/g). Preferably, the average surface area mass ratio of the particles is equal to or greater than 5 m²/g or 10 m²/g.

In accordance with an exemplary embodiment of the claimed invention, the lozenge comprises at least 70 weight percent beta cyclodextrin, at least 80 weight percent citrus bioflavonoid 50% HPLC, or at least 90 weight percent beta cyclodextrin.

In accordance with an exemplary embodiment of the claimed invention, the binder comprises at least one of the following: hydroxypropyl cellulose (HPC), hydroxypropyl methyl cellulose (HPMC), sodium alginate, alginic acid, guar gum, acacia gum, xanthan gum, carbolpol, cellulose gum (carboxymethyl cellulose), ethyl cellulose, maltodextrin, PVP/VA, povidone, one or more of microcrystalline cellulose, starch (partially or fully pregelatinized starch) and methyl cellulose.

In accordance with an exemplary embodiment of the claimed invention, the lozenge comprises one or more disintegrants and/or one or more lubricants. Preferably, the disintegrant is at least one of the following: microcrystalline cellulose, croscarmellose sodium, crospovidone, sodium starch glycolate, and starch. Preferably, the lubricants is at least one of the following: magnesium stearate, calcium stearate, sodium stearyl fumarate, polyethylene glycol (molecular weight greater than 3350), sodium lauryl sulfate, talc, mineral oil, leucine, and one or more of poloxamer. In some embodiments, the lozenge comprises about 65% to 92% beta cyclodextrin, about 4.5% to 30% binder, and 0.5% to 3% lubricant. The binder having disintegrant properties and preferably, being a pregelatinized starch.

In accordance with an exemplary embodiment of the claimed invention, the lozenge comprises about 65% to 92% beta cyclodextrin, about 4.5% to 30% binder, about 1.5% to 15% disintegrant and 0.5% to 3% lubricating agent.

In accordance with an exemplary embodiment of the claimed invention, lozenge comprises microcrystalline cellulose, pregelatinized starch, and sodium stearyl fumarate. Preferably, the beta cyclodextrin can account for about 85 percent by weight, microcrystalline cellulose accounts for about 4 percent by weight, pregelatinized starch accounts for about 9 percent by weight, and sodium stearyl fumarate accounts for about 2 percent by weight. In the dissolution test according to the test method USP <711>, the lozenge may have about 10% to 60% beta cyclodextrin (dissolving in about 15 minutes), about 30% to 90% citrus bioflavonoids 50% HPLC (dissolving in about 30 minutes) and at least about 60% citrus bioflavonoids 50% HPLC (dissolving in about 60 minutes). In the dissolution test according to the test method USP <711>, at least 90% of the lozenge can be dissolved within 30 minutes. In the dissolution test according to the test method USP <711>, lozenges can show at least 90% dissolution within 60 minutes.

In the disintegration test according to the test method USP <701>, the lozenge may show a disintegration time of less than 30 minutes. In the disintegration test according to the test method USP <701>, the lozenge may show a disintegration time of more than 30 minutes.

In accordance with an exemplary embodiment of the claimed invention, lozenges can comprise about 100 mg cyclodextrin, about 125 mg citrus bioflavonoid 50% HPLC and zinc.

In accordance with an exemplary embodiment of the claimed invention, the LOD% (loss on drying) of the water in the lozenge is less than 20% (water weight per weight (w/w)). Preferably, the LOD% of the lozenge water is less than 15% (water w/w). More preferably, the LOD% of the lozenge water is less than 10% (water w/w).

In accordance with an exemplary embodiment of the claimed invention, as measured by the test method USP <711>, at least 80% of the claimed lozenge dissolves within 30 minutes.

In accordance with an exemplary embodiment of the claimed invention, the lozenge comprises a disintegrant. Preferably, the disintegrant is selected from one or more of microcrystalline cellulose, croscarmellose sodium, crospovidone, sodium starch glycolate, and starch.

In accordance with an exemplary embodiment of the claimed invention, the lozenge comprises a lubricant. Preferably, the lubricant is selected from one or more of magnesium stearate, calcium stearate, and sodium stearyl fumarate.

In accordance with an exemplary embodiment of the claimed invention, a method for preparing lozenges comprises mixing formula with one or more binders to form. In accordance with an aspect of the claimed invention, the aforesaid method comprises heating the lozenges to above 50° C. after making the lozenge.

In accordance with an exemplary embodiment of the claimed invention, the lozenges prevent viral and bacterial infections in the mouth that can lead to respiratory tract infections.

In accordance with exemplary embodiment of the claimed invention, the oral hygiene composition is an oral sanitizer with immune support for viral, bacterial and COVID-19 prevention in a form of lozenges comprising:
a) anti-bacterial and anti-viral formula, killing 99.9% by reducing viral load;
b) physiologically acceptable, weakens and kills virus in less than 30 seconds;
c) immune supporting ingredients;
d) that weakens the virus membrane and kills the virus in the form of lozenges;
e) that is safe and organoleptically tolerable in the oral cavity and has no significant side effects either orally or systemically; and
f) that controls malodor and the viral load of pathogens in the mouth when it contacts the tissue of the oral cavity for a sufficient time to reduce the malodor, which also reduces the risk of COVID and other potentially harmful virus and viral pathogens comprising an effective antibacterial combination consisting essentially of:
   (1) from about 0.05% to about 0.2% by weight, based on the total weight of the composition, of sodium benzoate;
   (2) from 0% to Ethanol 190 proof 98.05;
   (3) from 0% to Blend of Thymol 0.295%, Menthol 0.1972, Methyl Salicylate 0.4348, Eucalyptus Oil 0.5663.

### Nasal Spray Device

In accordance with an exemplary embodiment of the claimed invention, a nasal spray device to deliver the claimed antibacterial and antiviral preventive composition with the immune support to the nasal cavity in metered doses. The nasal spray device comprises: a pressurized aerosol canister comprising a vial containing the claimed antibacterial and antiviral preventive composition with the immune support including an active ingredient, a propellant and, optionally, a co-solvent. The aerosol canister further comprises a metering valve having a valve stem and an actuator for the aerosol canister. The actuator comprises a stem block having a receptacle into which the valve stem of metering valve of the aerosol canister is received and axially located. The valve stem being displaceable relative to the vial of the aerosol canister to actuate the metering valve of the aerosol canister and a sump extending below the receptacle. The stem block further defining a discharge orifice for the claimed antibacterial and antiviral preventive composition with the immune support and a transfer channel through which a dispensed dose of the claimed antibacterial and antiviral preventive composition with the immune support is able to pass from the sump to the discharge orifice.

The fact that SARS-CoV-2 is vulnerable in nasal cavity, in accordance with an exemplary embodiment of the claimed invention, the nasal spray is provided comprising the claimed antibacterial and antiviral preventive composition with the immune support. In accordance with an aspect of the claimed invention, the nasal sprays comprise oxidative agents, such as beta cyclodextrins and citrus bioflavnoids to reduce the salivary load of nasal microbes. The modified sugar molecules attract viruses before irreversibly inactivating them.

In accordance with an exemplary embodiment of the claimed invention, by disrupting the outer shell of a virus, the claimed nasal spray destroys the infectious particles by simple contact rather than just blocking the viral growth. The claimed antibacterial and antiviral preventive composition with the immune support comprising BCDs and citrus bioflavanoids reduces the viral load in the nasal cavity and prevents the viral transmission via the nasal route.

The claimed nasal spray is a locally administered delivery system that lowers the SARS-CoV-2 viral load and reduce the nasopharyngeal microbiota, which tends to coat the surface aerosol particles and droplets during coughing or sneezing.

The claimed nasal spray lowers the pH level in the nasal cavity, thereby lowering the transmissions of virus spreading through the nasal cavity to respiratory tract which reduces the likelihood of infection. Moreover, adjunct therapy with oral and nasal spray when used in conjunction is more effective in reducing the viral infection and transmission of harmful pathogens to the respiratory tract.

In accordance with the exemplary embodiment of the claimed invention, the nasal spray pharmaceutical formulation comprises:
(a) an aqueous suspension having about 0.005% to about 5% by weight beta cyclodextrin and Citrus Bioflavanoids having the following particle size distribution profile:
(b) about 10% of the drug substance particles have a particle size of less than 0.90 microns;
(c) about 25% of the citrus bioflavonoid 50% HPLC particles have a particle size of less than 1.6 microns;
(d) about 50% of the citrus bioflavonoid 50% HPLC have a particle size of less than 3.2 microns;
(e) about 75% of the citrus bioflavonoid 50% HPLC have a particle size of less than 6.2 microns; and
(f) about 90% of the citrus bioflavonoid 50% HPLC particles have a particle size of less than 10 microns.

In accordance with an exemplary embodiment of the claimed invention, the aforesaid nasal pharmaceutical formulation comprises about 0.01% to about 1% by weight of the beta cyclodextrin.

In accordance with an exemplary embodiment of the claimed invention, the aforesaid nasal pharmaceutical formulation comprises about 0.04% to about 0.050% by weight of the beta cyclodextrin.

In accordance with an exemplary embodiment of the claimed invention, the aforesaid nasal pharmaceutical formulation is deliverable via a metered dose spray pump.

In accordance with an exemplary embodiment of the claimed invention, ambient air is a propelling agent for delivering each spray of the aforesaid metered-dose spray pump.

In accordance with an exemplary embodiment of the claimed invention, each spray of the aforesaid metered-dose spray pump delivers at least about 1 mcg of beta cyclodextrin.

In accordance with an exemplary embodiment of the claimed invention, each spray of the aforesaid metered-dose spray pump delivers about 1 mcg to about 100 mcg of beta cyclodextrin.

In accordance with an exemplary embodiment of the claimed invention, the aforesaid nasal pharmaceutical formulation further comprises a preservative, suspending agent, wetting agent, buffer, diluent, or combinations thereof.

In accordance with an exemplary embodiment of the claimed invention, the aforesaid nasal pharmaceutical formulation further comprises one or more of the following compounds: (a) microcrystalline cellulose; (b) carboxymethyl cellulose sodium; (c) dextrose; (d) benzalkonium chloride; (e) polysorbate 80; and (f) phenylethyl alcohol.

In accordance with an exemplary embodiment of the claimed invention, the aforesaid nasal pharmaceutical formulation is suspended in a suspending fluid comprising water, alcohol, glycol, or combinations thereof.

In accordance with an exemplary embodiment of the claimed invention, the aforesaid nasal pharmaceutical formulation further comprises an emulsifying agent, wetting agent, suspending agent, or combinations thereof.

### Mouth Strips

In accordance with an exemplary embodiment of the claimed invention, the claimed antibacterial and antiviral preventive rinse and/or spray with immune support is in the form of mouth or film strips. The mouth strips can be absorbed from oral mucosa. The claimed mouth strip prevents and treats diseases in a short time, by being absorbed rapidly when it is applied from the oral mucosa at lower dosages than other forms of dosage delivery.

In accordance with an exemplary embodiment of the claimed invention, a dissolvable film strip comprises the claimed antibacterial and antiviral preventive composition with the immune support that kills viruses and bacteria in mouth.

In accordance with an exemplary embodiment of the claimed invention, the claimed mouth strips are mouth soluble or dispersible, suckable, eatable, chewable, rapidly disintegrating in the mouth. The claimed mouth strips instantly freshens the subject's breath by killing 99.9% of the germs and bacteria, and reducing the viral load in the subject's mouth. The claimed mouth strips dissolve instantly in oral cavity and kill 99% of germs in seconds.

In accordance with an exemplary embodiment of the claimed invention, the claimed composition of the orally-absorbable mouth/film strip is freeze dried by means of lyophilization and maltotriose polymers, known as melatonin powder, are mixed with distilled water in an amount between 0.001-0.5 g/ml. The obtained mixture is poured over a smooth flat and non-adhesive layer in the form of a thin film. The poured film is freeze dried in a drying oven at relative humidity of 50% and room temperature overnight. The obtained film is cut thinly. Eventually, a natural component-maltotriose complex is obtained in the form of a thin film. The obtained film strip is elastic, adhesive and easily orally-dissolvable.

In accordance with an exemplary embodiment of the claimed invention, the claimed composition of the orally-absorbable mouth/film strip comprising citrus bioflavonoids is freeze dried by means of lyophilization. The citrus bioflavonoid powder is mixed with maltotriose polymer providing an amount of beta cyclodextrins that a person can take. Consequently, citrus bioflavonoid and beta cyclodextrin-maltotriose complex is obtained. Oral bioavailability of the citrus bioflavonoid HCL 50% in powder form is low however its absorption from the mouth increases by the changes made in the formulation. A higher level of absorption is provided when beta cyclodextrin is applied orally in the form of a film strip, in comparison with the absorption from gastrointestinal system. In accordance with an exemplary embodiment of the claimed invention, the film strip is in the structure of the beta cyclodextrin-citrus bioflavanoid 50% HPLC maltotriose complex.

In accordance with an exemplary embodiment of the claimed invention, a film or mouth strip comprises elastic, adhesive and orally-dissolvable natural components and obtained by performing the processes of mixing the natural component freeze dried by means of lyophilization and maltotriose polymer with water, pouring the mixture in the form of a thin film, drying the poured film.

In accordance with an exemplary embodiment of the claimed invention, in the aforesaid film strip, the mixture ratio of natural component-maltotriose polymer is in the range of 0.001-0.5 g/ml.

In accordance with an exemplary embodiment of the claimed invention, in the aforesaid film strip, the natural component lyophilized is beta cyclodextrin and citrus bioflavonoids 50% HPLC which reduces bacteria and virus in the mouth in less than 30 seconds.

In accordance with an exemplary embodiment of the claimed invention, the mouth or film strip comprises:
a) anti-bacterial and anti-viral formula, killing 99.9% by reducing viral load;
b) that is physiologically acceptable, weakens and kills virus in less than 30 seconds;
c) liquid and powder immune supporting ingredients;
d) that weakens the virus membrane and kills the virus in mouth strip form ;
e) that is safe and organoleptically tolerable in the oral cavity and has no significant side effects either orally or systemically, and
f) that controls malodor and the viral load of pathogens in the mouth when it contacts the tissue of the oral cavity for a sufficient time to reduce the malodor; and additionally that reduces the risk of COVID and other potentially harmful virus.

Research suggests a link between poor oral hygiene and severity of Covid-19 which stresses the importance of having a healthy mouth with less pathogenic bacteria and and viral pathogens

In accordance with an exemplary embodiment of the claimed invention, the mouth/film strip comprises an effective antibacterial combination consisting essentially of:
(i) about 0.05% to about 0.2% by weight, based on the total weight of the composition, of sodium benzoate;
(ii) from 0% to Ethanol 190 proof 98.05; and
(v) from 0% to blend of thymol 0.295%, menthol 0.1972. methyl salicylate 0.4348, Eucalyptus Oil 0.5663.

The claimed antibacterial and antiviral preventive rinse and/or spray with immune support protect against COVID-19 infection by killing the coronavirus before it can infect human cells.

Coronaviruses belong to the class of ‘enveloped viruses’, meaning they are covered by a fatty layer that is vulnerable to certain chemicals. The claimed antibacterial and antiviral preventive rinse can destroy the outermost layer or ‘envelope’ of the virus, preventing its replication in the mouth and throat. In particular, the claimed oral hygiene composition disrupts the outer lipid membrane, known as the ‘viral envelope’ or ‘lipid envelope’ of the SARS-CoV-2 virus, which causes COVID-19. The lipid envelope helps several viruses, including coronaviruses, bind to human cells while avoiding the host immune system.

Specific spike proteins called ‘glycoproteins’ on the surface of the envelope identify and bind to receptor sites on the host's cell membrane, which allows infection. The claimed antibacterial and antiviral preventive rinse can potentially modify the ability of the spike glycoproteins to interact with receptors on host cells. The lipid envelope does not change when viruses mutate, meaning the claimed antibacterial and antiviral preventive rinse can still work against any new coronavirus strains that emerge from this pandemic.

The development of antiviral drugs can be hampered by formulation challenges, most notably poor aqueous solubility of the active compound. Adequate drug solubility is imperative to ensure bioavailability and consequently, the efficacy of oral antiviral treatments. In the case of parenteral therapy, which offers the benefit of rapid onset in critically-ill patients, drug solubility is even more critical, given that intravenous solutions must be particulate-free and buffered to physiological pH. Cyclodextrin drug delivery systems can effectively overcome formulation challenges of antiviral drugs by offering improved solubility and bioavailability. HPβCD is cited in the FDA's list of Inactive Pharmaceutical Ingredients and is approved for use in oral and parenteral formulations due to its high aqueous solubility and excellent safety profile even at relatively high doses.

The 2019-nCoV is spreading rapidly across the globe and effective treatments are in urgent need. Companies are accelerating their drug development to combat 2019-nCoV infection; nevertheless, formulation development for any drug candidate is critical and challenging. HPβCD can effectively act as a safe, enabling excipient for solubility enhancement of antiviral drugs, stability improvement of therapeutic monoclonal antibodies, and as a vaccine adjuvant. In accordance with an exemplary embodiment of the claimed invention, the claimed antibacterial and antiviral preventive rinse and/or spray with immune support comprising cyclodextrins can be used for infection containment or as virucidal agents after structural modification.

Although the present invention will be described by way of several embodiments thereof, it should be realized that many alternatives, modifications, and variations will be apparent to those skilled in the art of the foregoing description. Accordingly, it is intended to embrace all such alternatives, modifications, and all variations as falling within the spirit and broad scope of the claims.

1. An oral sanitizer composition with immune support for viral, bacterial and COVID-19 prevention, comprising: 0% to 0.50% by weight of polysorbate 20; 0% to 10% by weight of Xylitol; 0% to 5% by weight methylated β-cyclodextrin; 0% to 10.50% by weight of vitamin C with citrus bioflavonoids; 0% to 10% by weight of sucralose; 0% to 0.10% by weight potassium sorbate; 0% to 0.006% by weight of sodium benzoate; 0% to 5% by weight of xanthan gum; 0% to 5% by weight of menthol; 0% to 5% by weight of zinc chloride; 0% 5% by weight of zinc masker; 0% to 10% by weight of FDC Green Color; and a remaining percentage by weight of water to total 100%.

2. The oral sanitizer composition of claim 1 formulated into a mouthwash, a mouth rinse, a mouth strip or a throat lozenge.

3. The oral sanitizer composition of claim 1 formulated into a mouth spray, a throat spray or a nasal spray.

4. The oral sanitizer composition of claim 3, wherein the oral sanitizer composition is deliverable via a meter dose spray pump.

5. An oral sanitizer composition with immune support for viral, bacterial and COVID-19 prevention, comprising: 0% to 0.50% by weight of potassium sorbate; 0% to 10% by weight poloxamer 407; 0% to 5% by weight of a buffering agent capable of buffering the oral sanitizer composition to a pH of 3.0 to 8.0; 0% to 10.5% by weight of a glycerin; 0% to 10% by weight of xylitol or sucralose; 0% to 0.10% by weight of zinc chloride; 0% to 0.006% by weight of FDC Green 3; 0% to 5% by weight of methylated beta cyclodextrins; 0% to 10% citrus bioflavonoids; 0% to 0.50% by weight of zinc chloride; 0% to 10% by weight of vitamin C; 0% to 25% by weight of ethanol 190 proof 98.05; 0% to 0.295% by weight of thymol; 0% to 0.1972% by weight of menthol; 0% to 0.438% by weight of methyl salicylate; 0% to 0.5663% by weight of eucalyptus oil; and a remaining percentage by weight of water to total 100%.

6. The oral sanitizer composition of claim 5 formulated into a mouthwash, a mouth rinse, mouth strip or a throat lozenge.

7. The oral sanitizer composition of claim 5 formulated into a mouth spray, a throat spray or a nasal spray.

8. The oral sanitizer composition of claim 7, wherein the oral sanitizer composition is deliverable via a meter dose spray pump.
